(19) United States
(12) Patent Application Publication
(10) Pub. No.: US 2005/0213037 A1
(43) Pub. Date: Sep. 29, 2005

(54) Method and apparatus of cornea examination

(76) Inventors: Erkin Abdullayev, Wichita, KS (US); G. Scott Bryan, Wichita, KS (US)

Correspondence Address: Bradley P. Sylvester;Suite 300, 200 North Broadway, Wichita, KS, 67202, US

(21) Appl. No.: 10/810,428

(22) Filed: Mar. 29, 2004

(51) Int. Cl. A61B 3/14; A61B 3/10; A61B 3/00

(52) U.S. Cl. 351/221; 351/245

(57) ABSTRACT

This invention comprises an improvement on the examination and evaluation of donor corneas. The improved apparatus includes the incorporation of a diffused light source, or sources, which are placed posteriorly to a donor cornea, with a viewing means placed anteriorly to said cornea. The apparatus involves the addition of the diffused light sources to existing apparatus, as well as the possible incorporation of a digital video camera or other electronic viewing means, which was not usable under the prior art. The method of placement of the diffused light source or sources is also novel, as compared with the prior art. Using this apparatus and method, the diffused light passes through the cornea, and is received by the viewing means. The viewing means is able to capture and image of the cornea, where the diffused light denotes any imperfections to the cornea. Use of the diffused light allows a complete view of the entire cornea at one time. The view of the cornea may be captured using a digital video camera or other means, with the image capable of being transmitted to another viewer, or where the entire examination can be done using a high resolution monitor.

### DISCLOSURE OF PRIOR ART

Prior art dealing with corneal investigation has previously dealt primarily with the examination of intact corneas in living patients. The investigation of corneas that are from hosts, being post mortem procurements, have been reviewed using what is called a slit lamp, that shines a bright beam of light onto the front side of the cornea.

Referring now to U.S. Pat. No. 4,187,005 (Rosenberger), a system for positioning a corneal endothelium microscope is shown. A flash system is discussed, but all light must originate in front of the eye. This system is intended for use with a living subject, and both the apparatus and the method are not suitable for determining corneal imperfections on subject corneas that are being examined for possible transplants.

Referring now to U.S. Pat. No. 4,189,215 (Humphrey), a method for positioning a patient's eye is disclosed, where a positioning beam is used to move a patient's cornea to a optimal position for eye examination. Again, this invention is only suitable for examination of a living eye. All light originates outside of and in front of the cornea and eye.

Referring now to U.S. Pat. No. 4,231,638 (Peck), a microscope that is used to observe cornea endothelium cells is disclosed. The cornea thickness is able to be determined with this invention. This patent neither describes or appears useful to investigate the entire cornea for aberrations or damage which would make the cornea unsuitable as a transplant option.

Referring now to U.S. Pat. No. 4,257,687 (Kohayakawa), a range finding device is described, using light that originates in front of the eye. This particular patent is useful with regard to achieving optimal photographic depictions of the eye being examined. It does not provide the ability to locate or determine damage to a cornea.

Referring now to U.S. Pat. No. 4,353,618 (Hagner et al.) an arrangement for examining a person's cornea using two distinct light beam paths is shown. This invention assists in the examination of the inner layers of a person's cornea, while the person being examined is still alive. This patent focuses on the reduction of reflection of light, rather than the use of light reflection to provide more detailed information. The use of several optical elements are depicted and shown, but which would not be workable with the presents apparatus and method disclosed in the discussion of the present in invention.

Referring now to U.S. Pat. No. 4,861,155 (Downey), a method for determining the curvature of the cornea is shown. This particular patent is more concerned with overall cornea shape, rather than imperfections within the cornea. Further, imperfections due to surgery would likely not be discoverable under this particular patent method.

Referring now to U.S. Pat. No. 5,535,743 (Backhaus et al.), an apparatus to examine the aqueous humour in an anterior chamber of a person's eye is shown. Although two separate light paths are discussed, they again must be from the front external side of the person's eye, and as FIGS. 1 and 2 show, very little attention is given to the cornea. In fact, the light in this invention is intended to pass at a virtual right angle through the cornea, rather than through an angled approach.

Referring now to U.S. Pat. No. 5,475,451 (Robert et al.), an improvement in the examination of a patient's eye is shown. Use of this invention with a donor cornea is discussed, but it has to do with the position of the apparatus relative to the eye, rather than an investigation into the likelihood of cornea damage. This invention contemplates that the cornea has been previously reviewed and found satisfactory for transplant use.

Referring now to U.S. Pat. No. 5,706,072 (Kawamura), a measuring apparatus is shown in which multiple beams of light are used to assist in measuring a person's physical structure of the eye. This apparatus, and method do not include or contemplate the examination of the person's eye for purposes of determining suitability of a cornea for transplant after having been removed from a donor.

Referring now to U.S. Pat. No. 6,575,573 (Lai et al.) a recent method to make a corneal profile is shown. This invention relies heavily on the thickness and curvature measurements that are obtained with this invention. The topography of a cornea is the desired results achieved under this patent. There does not appear to be any real use of this invention to examine a person's eye for all types of corneal damage. Since damage from surgery is often below the outer epithelial layer of a cornea, it is detectable under this patent only if surgery has caused the deformation on the external curvature of a cornea.

Referring now to U.S. Pat. No. 6,578,964 (Otten III et al.), the examination of the cornea for transplant purposes is described. In this invention, a beam of collimated light passes through the target cornea to produce a distorted wavefront. By analyzing the wavefront, modifications to the cornea can be determined. One of the benefits of this invention is that it reduces human error as long as the cornea and equipment are placed in proper relationship to each other. One of the drawbacks of this invention is that the actual problems with the cornea may not be completely discernible without having a person physically observe the modifications. The present invention allows a person to physically observe the modifications to the cornea without having to interpret wavefronts.

Referring now to U.S. Pat. No. 6,588,902 (Isogai), an apparatus is shown which uses a beam of light to assist in centering a person's eye for examination. This allows the observer to detect the apex of the cornea, but not the extent of damage done through surgery and or other deterioration.

Referring now to U.S. Pat. No. 6,588,903 (Rathjen), a method used to measure reflected light rays to determine the thickness of a person's cornea is shown. This invention is to assist in examination of the cornea prior to LASIK surgery, and is not useful for detecting the type of damage that has previously occurred to a cornea through other types of injury or neglect.

Referring now to U.S. Pat. No. 6,607,273 (Sarver et al.), multiple light beams are used to measure the shape of a cornea using the reconstruction of light reflection to determine the actual curvature. A computer is necessary to process the image in order to apply a reconstruction algorithm. If there are any benefits under this invention to detect problematic attributes with a cornea, a computer system is apparently still necessary to analyze the data obtained through the reflective light rays. Therefore, actual problems with a cornea under this invention was still require further personal analysis by a human being to determine their full extent.

### FIELD OF THE INVENTION

This invention relates to an improved method and apparatus to analyze a donor cornea that has been removed from the host, placed into viewing chamber, and is being evaluated for possible transplant. Observations of the cornea are able to be made with accuracy and precision using a plurality of directed light beams that originate behind the cornea, and which shine through the cornea. The light is of a diffused nature, and passes through the cornea from the back to front, and is viewed using a stereo microscope. The view of the donor cornea in this manner provides a suitable and reliable method to eliminate various imperfections. Damage done through surgery, as well as through neglect of the cornea prior to removal are readily seen.

### BACKGROUND OF THE INVENTION

The transplanting of corneas occurs with great frequency. Typically, a person removes a cornea from a host, careful to leave surrounding tissue intact, and the cornea is transported to an examination facility where the cornea can be evaluated for possible transplant. There are numerous problems with corneas that make them unsuitable for transplant. Epithelial tears or abrasions can occur through trauma. Scars from previous penetrating traumas (stroma) can also adversely affect a cornea's suitability for transplant. Where the eye has been exposed to air for an abnormally long period of time, such as in a situation where the donor was unconscious while their eyes were open, the external later of the cornea will often dry, and can result in a change in the consistency of the cells in this external layer. Such a change can often cause a different optical quality, and one that is not suitable for transplant.

Refractive (LASEK) surgery, and other related surgical procedures, involve necessary scarring of the cornea, which are generally detrimental attributes for a cornea intended for transplant. Typically, this type of surgery involves making a circular slit in the epithelial layer, and removing a portion of the middle layer of the cornea. Refractive surgery causes a permanent change in the shape of the cornea. While the donor may have benefited greatly from such an operation, the recipient of such a transplant cornea effectively receives a misshapen cornea that does not properly direct light into the eye.

If the refractive surgery was skillfully done, it may be extremely difficult, even with the slit lamp, to determine the prior history of the surgical procedure. If a person has had refractive surgery, their cornea is generally unacceptable for transplant. In the past, many people have received corneas affected through previous refractive surgery, simply because the evaluation means did not readily disclose the history of such surgeries. It is critical that corneas be evaluated properly prior to transplant to avoid having the recipient receiving a defective cornea.

Prior art has primarily utilized a slit lamp to evaluate the cornea to determine suitability for transplant. After the cornea is removed from the host, it is placed into a storage container that allows visual examination of the cornea. Utilization of a slit lamp has been the preferred method for many years, where the slit lamp comprises a bright beam of light that is directed to the exterior surface of the cornea. The slit lamp projects a linear beam that illuminates a thin strip of the cornea from top to bottom. The observer must rely on the information that is reflected from a single slit lamp beam played over a small strip of the total corneal area. One of the inherent drawbacks to this technique is that relying on the intense level of reflected light makes it extremely difficult to determine some of the more subtle problems with a cornea that are effectively the most problematic, for example detecting prior surgical involvement. A slit lamp has usefulness in detecting dryness damage on a cornea, but the band of light presents drawbacks to making an adequate determination quickly and easily. Since the slit lamp only illuminates a very small fraction of the total cornea surface area, a mapping of the observable problems with a cornea becomes difficult, since a total picture must be visually pieced together through multiple observations of the slit lamp beam as it plays across the surface of a cornea. Surgical involvement is difficult to detect using a slit lamp.

Another drawback to the slit lamp method is that recordation of the quality of the cornea, using a camera is difficult, including situations where digital cameras are used. Digital cameras offer the ability to supply images electronically, which are then capable of being transmitted to other parties.

The primary problems with use of a slit lamp beam for examination purposes is that is involves such a strong and intense light. Even when reflecting the true extent of imperfections, it is difficult to see because the light is so bright, and subtle imperfections in the cornea are difficult to differentiate from the glare. Coupled with this problem is the fact that a single photograph of a given point in time, using a slit lamp evaluation, only reveals a very narrow strip of the total cornea. A complete evaluation of a cornea, using a slit lamp method, requires multiple pictures taken through multiple examination points in time, comprising the various points in time when the slit lamp was moving across the cornea. This is a mentally challenging compilation to assess all photographs from their individual status into one to create a complete picture, and if a camera is used, multiple image transfers are required for a second party.

### SUMMARY OF THE INVENTION

This invention comprises a method and suitable apparatus for the complete investigation and examination of a cornea that has been removed from a host, and which is intended to be used for transplant. Such corneas are placed into storage containers that both protect the cornea and position it for examination. The cornea has a front convex surface, with the rear side comprising a concave surface. The improved method of examination comprises the placing of one or more diffused light sources behind the cornea. Light should be diffused, or filtered, and not direct. Where multiple light sources are used, each light source used impacts the cornea at an angle different from the other light source. The light source must be diffused, in relation to the cornea for all light sources, to fully appreciate this improved method. If a slit lamp is used in conjunction, this method also allows improved examination techniques of the slit lamp examination, but often detracts from the diffused light method quality.

Typically one light source is placed at an angle behind the cornea. If a secondary light source is used, it is also positioned behind the cornea, but at a different angle as compared to the first light. Typically, the distance between the two lights is no more than 5 to 6 inches. The first light will enter the cornea at an angle approaching a right angle. Light from the second source will enter the cornea at very shallow angle, since the second light source is just behind, and immediately to one side of the cornea. Both of these light sources are not put into direct line of sight with the observer viewing the cornea, but are diffused, to the observer. This can involve diffused reflected light, or filtered light. In this way, the light viewed by the observer will not involved direct glaring rays, as is consistent with the more intense direct light.

As both light rays move through the cornea, they encounter distortions as a result of scar tissue, or other types of undesirable features within or on the surface of the cornea. The light beams are scattered and deflected slightly, due to imperfections in the cornea.

Unlike the slit lamp, which requires an observation of all reflected light, including that reflected from normal areas of the cornea, this method allows observation of cornea imperfections using virtually only the light that has been reflected from the cornea imperfections. While there is some minor reflection of light from the normal areas of the cornea, this is at an extremely low level. Both diffused light sources may be moved, to take advantage of optimal viewing conditions.

Another advantage of using this method, is that an apparatus is available by simply adding minor modifications to common apparatus already typically used. Another advantage to this method and apparatus is that digital photography and videography are permissible, since the overall intensity of light is lower. This allows review by another party, that is not able to be physically present with the cornea being reviewed. Further, the data acquired through digital photography and videography can be cataloged, and shown at a later time. This increases the ability of the evaluator to both determine with greater accuracy imperfections in a donated cornea, as well as providing information to others regarding the status of the cornea imperfections.

### DESCRIPTION OF THE DRAWINGS

FIG. 1 is a perspective view of the typical apparatus used to evaluate and examine corneas, including the typical slit lamp, a stereo (binocular) microscope, along with multiple diffused light sources, and an incorporated video digital camera.

FIG. 2 is a cross sectional view of a cornea as it appears while still invested in a donor's eye.

FIG. 3 is a partial depiction of a cornea shown as a cross-sectional view.

FIG. 4 depicts a top view of typical cornea and surrounding sclera tissue, such as would typically be seen after it was removed from a donor, and depicts the typical view seen during examination purposes.

FIG. 5 depicts a cornea, having a circular refractive surgery scar indicated on the surface of the cornea.

FIG. 6 depicts a cornea with the area highlighted by a slit lamp beam that has been directed upon it, indicating the viewable area illuminated with the slit lamp.

FIG. 7 depicts a diagram view of a cornea being evaluated with a microscope and slit lamp for illumination.

FIG. 8 is a diagram showing the path of diffused light passing through a cornea, being received by a viewing microscope and digital video camera, with the image received by the digital camera able to be transmitted to a viewing monitor.

FIG. 9a depicts a cornea as it would appear when viewed by a slit lamp, also showing the intensive glare in the central portion of the cornea from the slit lamp.

FIG. 9b depicts a cornea as it would appear when viewed by a slit lamp, indicating prior surgery as noted by the circular scar.

FIG. 10a depicts a cornea as it would appear when viewed by a slit lamp, showing wrinkles on the surface of the cornea.

FIG. 10b depicts a cornea as it would appear when viewed by the improved method using diffused lighting, showing the surface of the cornea and all of the wrinkles and folds.

FIG. 10c depicts a cornea as it would appear when viewed by a slit lamp in combination with the improved method of using diffused light.

### DETAILED DESCRIPTION OF THE INVENTION

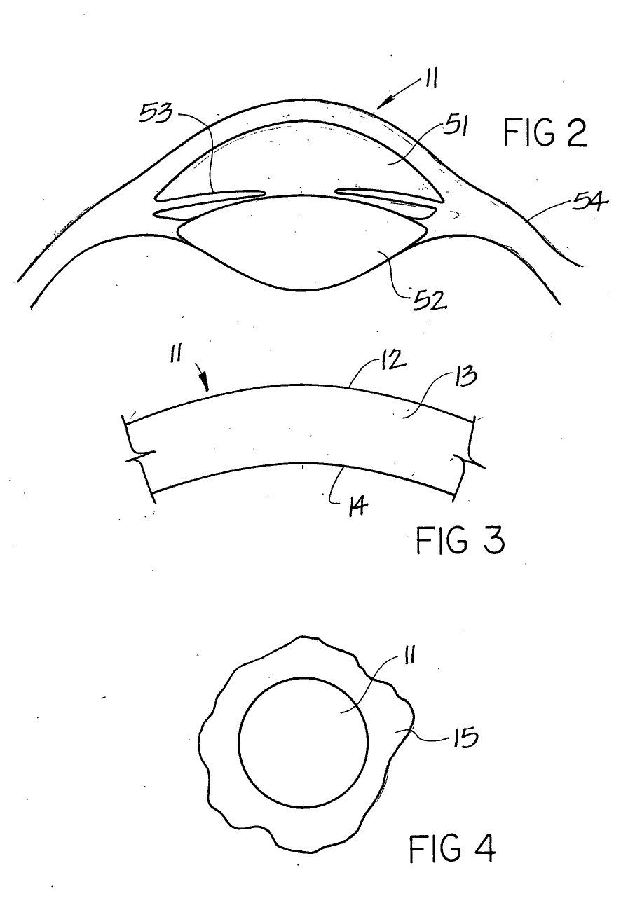

Referring now to FIG. 2, the cornea 11 is the transparent part of the external tunic of the eyeball. It is almost circular in shape, and may be slightly broader in the transverse band in the vertical direction. It is convex anteriorly, and projects forward from the sclerotic. The cornea 11 lies on top of an interior chamber 51, and in front of the lens 52 which is covered in part by the iris 53. The sciera 54 surrounds the peripheral edge of the cornea 11. The degree of curvature for each cornea 11 varies from individual to individual, as well as varies within the same individual during different stages of the life span. For example, a cornea 11 will tend to flatten out over time, so that an older person will exhibit a flatter cornea 11 than a younger person typically would.

Referring now to FIG. 3 The cornea 11 is comprised of multiple layers. Although each layer is technically separated by a membrane, where the membranes may be considered layers themselves, only the three principal layers are referred to in the drawings. The external first layer comprises several strata of epithelial cells 12, that are continuous with those of the conjunctiva. These layers of cells comprise a layer of columnar and polyhedral cells with a bottom layer of three or four layers of scaly epithelium with flattened nuclei. This layer of epithelial cells 12 is fairly thin.

Beneath this first layer of epithelial cells 12, and separated by a membrane from the first layer, lies a thick central fibrous structure. This is referred to as the substantia propria 13 or middle layer. This layer is perfectly transparent, and is also continuous with the sclerotic. It is composed of approximately 60 flattened lamellae, that are superimposed on top of each other. The fibers within this layer 13 are generally parallel with each other, although there are certain fibers of alternating lamellae that are positioned at right angles to each other.

Beneath layer 13 lies a homogenous elastic lamina 14, with a single layer of endothelial cells that form the lining membrane of the anterior chamber of the eyeball. This anterior layer 14 is also perfectly transparent, and does not presents a structure that is recognizable under a microscope unless there is damage to it.

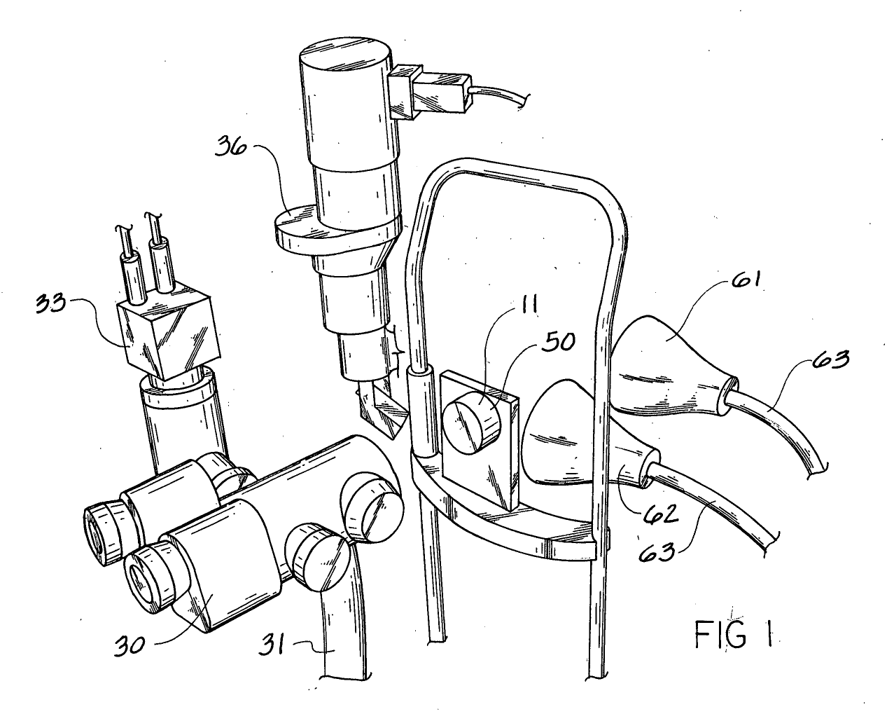

Referring now to FIG. 4, a cornea 11 is shown in the condition typical following removal from the host. Since corneas 11 are able to be transplanted from one individual to another, they are removed from the donor with a portion of the attached sclera 15, and placed in storage containers for review. An example of such a storage container is seen in FIG. 1, indicated as number 51.

Corneas 11 have a limited time in which they can be transplanted, with the review of the cornea 11 requiring both speed and accuracy. There are several primary imperfections looked for during the review of donated corneas.

The first type of imperfections involves a check to see whether epithelial sloughing has occurred. This can involve both the front exterior side 12, as well as the interior side layer 14 of the cornea 11. For example, the posterior elastic lamina layer 14 will curl or roll up when separated from the rest of the cornea 11. This can occur through trauma, or through faulty technique of removal in which an improper or insufficient amount of sclera 15 is attached, or where trauma has occurred physically to this layer 14.

Another type of imperfections for which the cornea 11 is reviewed comprise scar tissue from previous penetrating trauma. Scar tissue can comprise unintended damage to the cornea 11 as well as result from intended damage incurred through surgery. In either instance, scar tissue can adversely affect a cornea's 11 suitability for transplant.

Since corneas 11 are donated, they are not retrieved from living donors. A history of the cornea 11, with regard to prior surgery is generally not available. Further, the donor may have been unconscious prior to retrieval. Therefore there are a number of corneas 11 that are obtained from people that have suffered periods of unconscious states or comas. It is important that the cornea 11 has not been exposed to air for an abnormally long period of time, such as in a situation where the donor was unconscious while their eyes were open, while in a coma, or otherwise. Such prolonged exposure can affect the epithelial cells 12, due to a drying of the surface of the cornea 11, and result in a diminished optical quality. Such corneas 11 may not be suitable for transplant.

Perhaps most importantly, this method offers a heightened accuracy and ability to determine whether or not the donor has had refractive surgery. This type of surgery generally renders a cornea 11 unusable as a suitable transplant cornea. The evidence of such surgery is difficult to detect, and quite often donor records are not readily available to make this determination. In some instances even relatives are unsure whether or not the donor has ever had this type of surgery.

Refractive surgery dramatically effects the cornea 11 due to the nature and invasive procedure of the surgery itself. Referring now also to FIG. 5, a circular slit 21 is shown on the cornea 11, where the slit has been made through the epithelial layer 12 to create a flap which is then raised so as to expose the middle layer substantia propria 13. In such a surgery, the surgeon then removes a portion of the substantia propria 13 so as to decrease the thickness of the substantia propria 13 and modify the characteristics of the cornea 11. Since refractive surgery causes a permanent change in the shape of the cornea 11, the intended recipient of a donor cornea 11 generally is unable to take advantage of the modifications in the same manner. While the original donor may have benefitted greatly from such an operation, the recipient of such a transplant cornea effectively receives a misshapen cornea 11 that does not properly direct light into the eye, and the recipient may be even worse off than before the transplant.

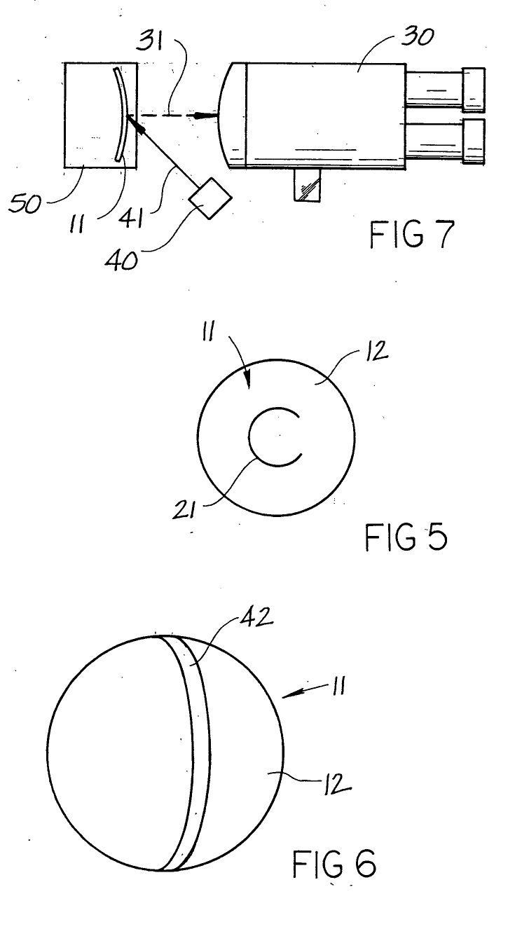

Referring now also to FIG. 6 and FIG. 7, the prior art method of reviewing a cornea 11 is shown. This is known as the “slit lamp” method. In this method, the cornea 11 is placed in a suitable holder 50, with a viewing microscope 30 having a direct line of sight 31 with the front or posterior side of said cornea 11. FIG. 1 depicts some of the typical apparatus used in this method, with the exception of light sources 61 and 62, or camera 33. The slit lamp light source holder 36 is shown in FIG. 1, which directs a thin band of light, that is played across the surface of the cornea 11 from what is typically referred to as a slit lamp 40. As FIG. 7 depicts, the slit lamp 40 directs an intense beam of light onto the surface of the cornea 11, illuminating a thin band 42 of a cornea 11, as exampled in FIG. 6, where the thin band of light 42 is shown on the cornea 11.

The person viewing the cornea 11, using the slit lamp method will be forced to rely on light that is reflected off of the surface layer 12 of the cornea 11, as well as light reflected from the lower layers 13 and 14. Moisture itself gives the cornea 11 a shiny appearance under intense light, such as that emitted from a slit lamp 40, and such shiny appearance will cause a great deal of light to be reflected to the viewing microscope 30. In order for the viewer to distinguish between imperfections and normal cornea 11, the light intensity must be significant. The intensity of light also causes increased glare. There is little distinction between the imperfections and ordinary reflection of slit lamp 40 light, giving rise to significant eyestrain and potential damage to an observer who makes multiple observations on a regular basis.

Referring now also to FIG. 7, an example of the interference of light upon itself is shown when using a slit lamp method. In this method, light rays 41 are emitted from slit lamp 40 and directed to the cornea 11. For example, this particular cornea 11 has an imperfection 44 in its middle layer 13. Light rays 41 strike the surface layer 12 of the cornea 11, with some of the light 31 reflected toward the viewing microscope 30. Not all light is reflected, and a portion continues along into the middle layer 13 of the cornea 11, where the imperfection is. The inherent problem here is that light rays 41 tend to interfere with other portions of the light 41, with regard to observations. Some of the reflected light rays 31 are unnecessary to view, yet may actually comprise some of the brightest light directed towards the viewing microscope 30, such as that comprising reflected glare due to moisture on the surface 12 of the cornea 11. Even in instances where the imperfection is on the outer epithelial layer 12, light rays 31 from unaffected areas will continue to interfere with regard to the discernment between unnecessary light rays and information carrying light rays.

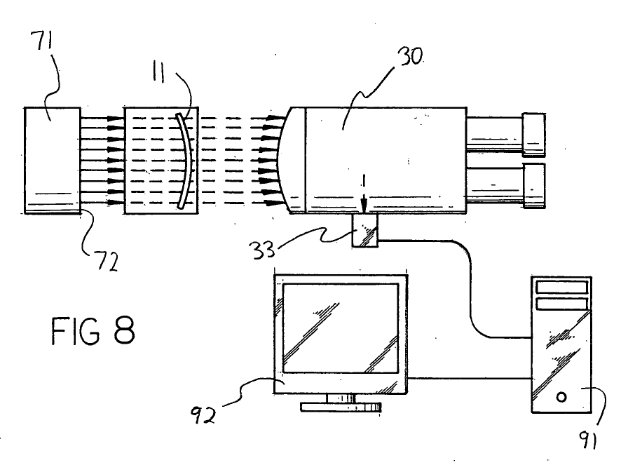

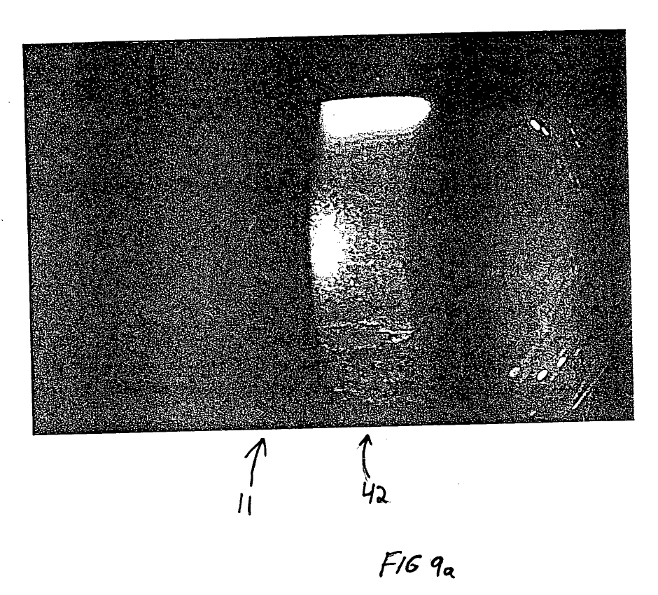

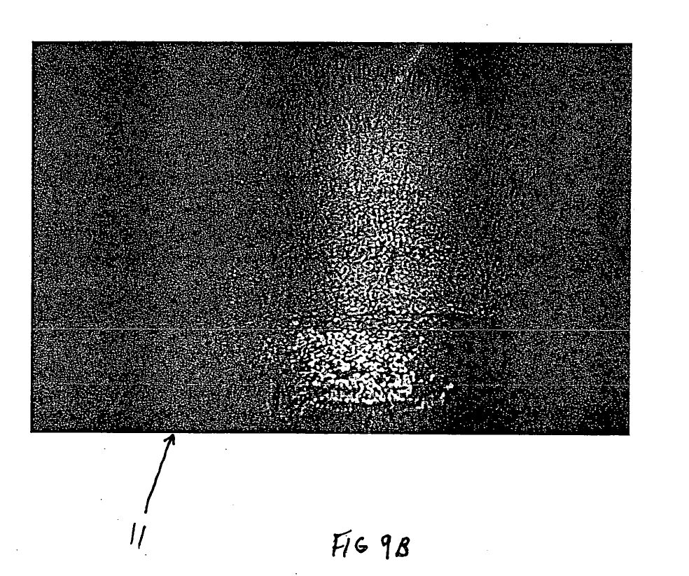

Referring now to FIG. 8, and also to FIG. 1, the improved method disclosed herein is shown. This method comprises the placement of a light source 61 behind the cornea 11, where said cornea 11 is fixed in position, so that light strikes the inner or posterior side of the cornea 11. FIG. 1 depicts multiple light sources 61 and 62, while FIG. 8 depicts only a single light source 71. In FIG. 8, the light source 71 has a means to diffuse the direct light, such as a filter 72, which allows the light source 71 to be directly behind the cornea 11. In FIG. 1, the diffusing of the light comes from it being reflected off of the interior of the shade of each light source 61 and 62, where the reflective qualities cause the light to be diffused.

In either light placement, as shown in FIG. 1 or FIG. 8, any light source provides a diffused light onto the back anterior side of the cornea, 11. Since the interior layer 14 is optically pure, the light moves through it with virtually no reflection. As light strikes any imperfection, part of light will be reflected. The light continuing to move through the epithelial layer 12 suffers little reflection problem, and follows a path that does not interfere with the viewing area of the microscope 30.

In this method, there is little unwanted light that is able to interfere with observations of the cornea 11 while being viewed through the microscope 30. In order to further emphasize the depth and configuration of imperfections, a second light source 62 may be used, as shown in FIG. 1. The second light source 62 emits diffused light toward the cornea 11 at a more shallow angle than the first light source 61. Light rays will move through the endothelial layer 14 without appreciable change, until they strikes an imperfection. The imperfection will cause a partial scattering of light and reflection of said light that would result in a portion of light being reflected toward their viewing microscope 30, defining the imperfection.

The use of a first and second light source 61 and 62, where both light sources are providing diffused light to the cornea 11, provide together the appearance of depth and clarity of imperfections not seen previously in any prior art. Both light sources 61 and 62 are not slit lamps 36, in that they do not need to project a thin band of intense light. The light sources 61 and 62 are typically low voltage light sources, and provide diffused light. Light sources 61 and 62 may simply comprise illuminating bulbs that emits a desired amount of light through a filter or through a reflective shade. The benefit to the examiner of the cornea 11 is that the examiner's eyes do not receive an intense level of light that is the result of a direct reflection from a slit lamp beam. This method therefore allows the examiner to protect the longevity of his eyes from degradation caused by constantly viewing intense light.

Referring now to FIG. 1, the typical apparatus used in the viewing a cornea 11 is shown. In this depiction, a viewing microscope 30 is shown, having a suitable mount or base 31 that allows the line of sight to be directed towards a cornea 11 that is fixed within a proper holder 50. The prior art slit lamp 36 is also shown on this standard configuration of prior art. A slit lamp may be used along with this improved method, but generally will detract from the quality of the view obtainable with diffused light.

Typical first light source 61 and second light source 62 are shown, positioned off to one side of the holder 50 opposite the viewing microscope 30. Optimally, light sources 61 and 62 should be flexible, being fixed on adjustable supports 63, so as to allow multiple positions of light source 62 and 61 to any desired location. Light sources 61 and 62 may be incorporated directly into the viewing microscope 30 and supporting assembly, or may be independently attached to a separate support, or even be independently attached to their own supporting bases.

As is also shown in FIG. 1, a camera assembly 33 is shown, that receives information from the viewing microscope 30, comprising a portion of the light entering into the viewing microscope 30 from the cornea 11. This comprises an optimal viewing means. Unlike the slit lamp method, this improved method does not overwhelm the ability of the camera to receive and properly record the data in a manner that it may be viewed digitally with the same appearance as the observer has through the viewing microscope 30.

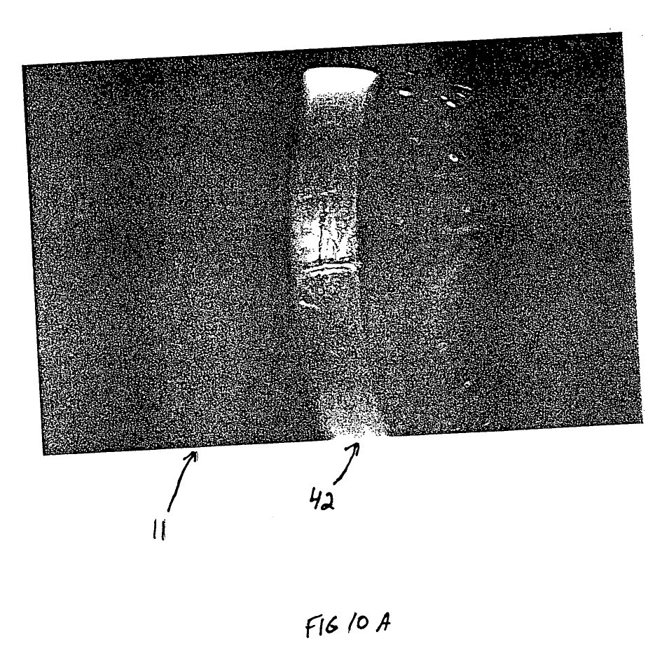

An example of the types of imperfections viewed with this improved method are shown in FIGS. 9a and 9b. FIG. 9a depicts what is viewed using a slit lamp method only. As is seen, the cornea 11 is fairly dark and unremarkable, with the exception of the intense band of light 42 projected by the slit lamp 40. As is seen in the area of illumination by the intense band of light 42, some type of imperfection is noted in the central part of the lighted area 42. Utilization of the improved method, in which the rear light sources 61 and/or 62 are used, is shown in FIG. 9b.

Another beneficial attribute, is that this method allows complete illumination and viewing of a cornea 11, which can be helpful in determining the type of damages or imperfections seen. Referring now to FIG. 9b, the same cornea 11 shown in FIG. 9a is depicted without the interfering light of the slit lamp 40, and is shown using light sources 61 and/or 62. Not only is the area of viewable information much greater in 9b, but clear evidence of refractive surgery is evident. The telltale circular scar 21, as also depicted and shown in FIG. 5, is evident now in FIG. 9b. This is clearly not evident in FIG. 9a, other than some type of minor surface imperfection. A person using this improved method, and seeing the depiction of FIG. 9b, can instantly determine that a particular cornea is not suitable for transplant. Using the old method, with the view shown in FIG. 9a, such a conclusion is not readily determinable. Further, interference with normal informational light is clear in FIG. 9a, as shown by the central glare within the band of light 42.

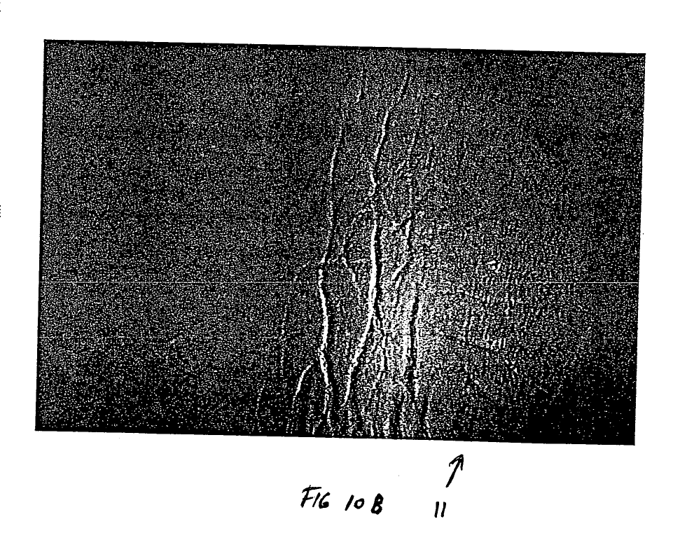

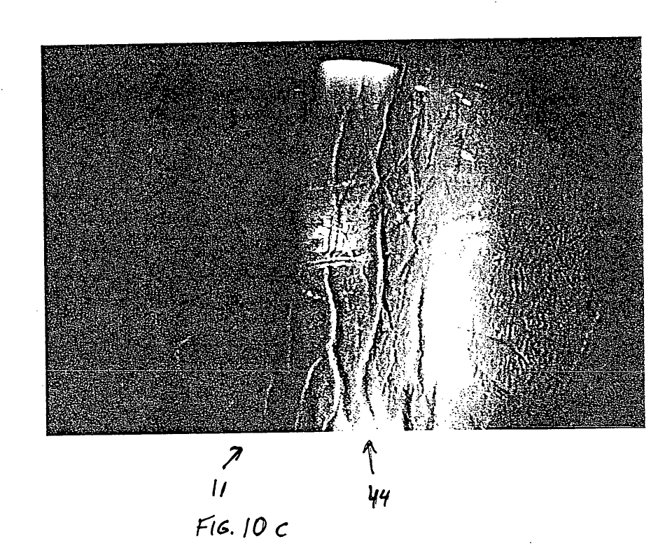

Referring now also to FIGS. 10a and 10b, the difference of the view of a cornea 11 using the slit lamp method and the improved method described is shown. FIG. 10a show the thin band of light 42 with a central glare, in which some central imperfection is noted. Very little information is obtainable as you move further from the center of the cornea 11 using the slit lamp method. The contrasting image, using the backlighting method, in which light sources 61 and/or 62 are used, is clearly evident in FIG. 10b. FIG. 10b allows viewing of the majority of the cornea 11, and shows multiple folds that render this cornea unsuitable for transplant. FIG. 10b further allows this information to be captured though digital imaging, since the level of light necessary is not excessive, with such information able to be transferred to other viewers with little loss of information. Such information is helpful both to the physician determining the actual suitability of the cornea 11 for transplant, as well as information to the person obtaining the cornea 11 from the donor.

Referring also to FIG. 10c, a view of the cornea 11 using both the slit lamp method and light sources 61 and 62 is shown. The interference caused by the slit lamp light 44 can be seen, along with information now provided by the backlighting light sources 61 and 62. When comparing FIGS. 10b and 10c, 10c shows very little increase in information when using a slit lamp in conjunction with backlighting light sources 61 and 62. In fact there is a loss of information as to the nature of the folds in FIG. 10c as compared to FIG. 10b, due to the interference of light from the slit lamp, and this particular view is unlikely to be usable for digital recordation.

Referring now to FIG. 1 and FIG. 8 the ability to magnify the viewing area, through such instruments such as a stereomicroscope 30 is used. A digital video camera 33 is incorporated into the stereomicroscope 30 in such a manner that it is able to receive the picture viewed through the stereomicroscope 30. The digital video camera 33 sends data, comprising digital information about the view of the cornea 11 to computer 91, or other similar data managing device, which then displays the picture recorded by the digital video camera 33 on a high resolution monitor 92.

The benefits of using a monitor 92 for examination of the cornea 11 is a dramatic benefit realized through this system and method. A clear view of the cornea 11 is able to be displayed on the monitor 92, without significant adjustments made for intense light. The same view would not be obtainable with a slit lamp 36, if one were used, since the intensity of the light received by the digital video camera 33 would be significant, and that resulting glare would likewise be displayed on the monitor 92.

With the advent of using a monitor 92 to view corneas 11 during the examination, such information in the form of digital images can be transmitted to other sources for multiple purposes. For example, information about the cornea 11 being examined can be sent to the person who obtained the cornea 11 from the cadaver, to better educate the person doing the collection on the quality of corneas being received. More importantly, the condition of the cornea 11 to be transmitted to a surgeon for their review prior to surgical transplant. Previously, the corneas were examined using a prior art techniques and slit lamp 36, with the person doing the examination suffering from significant eye fatigue, with very little ability to convey what they saw to any other individuals. The use of a digital video camera 33 makes information transfer possible using this arrangement and using diffused light. This also reduces eye fatigue and potential eye damage of the examiner, since the examiner no longer has to stare at bright reflected light during a lengthy examination process. The monitor 92 is also able to display the entire cornea 11, so that all imperfections can be seen at the same time. The prior art slit lamp method only allowed a strip of the cornea to be examined at a single time, this making this present invention a useful tool in the overall evaluation of an entire cornea 11 for purposes of approving it for transplant.

From the foregoing statements, summary and description in accordance with the present invention, it is understood that the same are not limited thereto, but are susceptible to various changes and modifications as known to those skilled in the art and we therefore do not wish to be limited to the details shown and described herein, but intend to cover all such changes and modifications which would be encompassed by the scope of the appended claims.

1. An improved apparatus for viewing donor corneas, comprising:
a. a means for holding a cornea;
b. at least one diffused light source, positioned anteriorly to the cornea, where said diffused light is capable of illuminating said cornea, as seen from the posterior side of said cornea;
c. a viewing means, capable of receiving the view of the cornea from the posterior side of said cornea, where the view of the cornea is obtained using said diffused light source.

2. An improved apparatus for viewing donor corneas, as recited in claim 1, in which the diffused light source uses a filter means positioned between the light emitting point, and the cornea.

3. An improved apparatus for viewing donor corneas, as recited in claim 1, in which the diffused light source is positioned in direct line of sight with the cornea and viewing means.

4. An improved apparatus for viewing donor corneas, as recited in claim 1, in which the diffused light sources comprise at least 2 light sources, which are positioned at different angles in relation to the posterior side of said cornea.

5. An improved apparatus for viewing donor corneas, as recited in claim 1, in which the viewing means comprises digital video camera, capable of receiving the diffused light and creating an electronic image.

6. An improved apparatus for viewing donor corneas, as recited in claim 1, in which the viewing means comprises a monitor, that receives an electronic image of the view of the cornea.

7. An improved apparatus for viewing donor corneas, as recited in claim 1, in which the diffused light sources are further combined with a slit lamp, where the diffused light sources are positioned anteriorly to the cornea, while the slit lamp is positioned anteriorly to the cornea.

8. An improved apparatus for viewing donor corneas, comprising:
a. a means for holding a cornea;
b. at least one diffused light source, positioned anteriorly to the cornea, where said diffused light is capable of illuminating said cornea, as seen from the posterior side of said cornea;
c. a microscope means to receive the view of the cornea, with an incorporated digital video camera, where said digital video camera is able to record and transmit the image of the cornea to a monitor.

9. An improved apparatus for viewing donor corneas, as recited in claim 8, in which the diffused light source uses a filter means positioned between the light emitting point, and the cornea.

10. An improved apparatus for viewing donor corneas, as recited in claim 8, in which the diffused light source is positioned in direct line of sight with the cornea and viewing means.

11. An improved apparatus for viewing donor corneas, as recited in claim 8, in which the diffused light sources comprise at least 2 light sources, which are positioned at different angles in relation to the posterior side of said cornea.

12. An improved apparatus for viewing donor corneas, as recited in claim 8, in which the diffused light sources are further combined with a slit lamp, where the diffused light sources are positioned anteriorly to the cornea, while the slit lamp is positioned anteriorly to the cornea.

13. A method of examining donor corneas, comprising the following steps:
a. placing a diffused light source on the posterior side of the cornea being examined, with the diffused light emitted from said source moves through the cornea from the posterior to the anterior side;
b. placing a viewing means on the anterior side of the cornea, where said viewing means is capable of receiving an image of the cornea, using the diffused light.

14. A method of examining donor corneas, as recited in claim 13, in which the diffused light source is placed directly behind the cornea, so that it is in a line of sight with the cornea and viewing means.

15. A method of examining donor corneas, as recited in claim 13, in which at least two diffused light sources are placed posteriorly to the cornea, so that each light source projects its emitted diffused light to the posterior side of the cornea at an angle different from the other light source.

16. A method of examining donor corneas, as recited in claim 13, in which the diffused light source uses a filter means to diffuse the light.

17. A method of examining donor corneas, as recited in claim 13, in which the light source uses a reflective surface that diffused the light upon reflection.

18. A method of examining donor corneas, as recited in claim 13, comprising the additional step of placing a slit lamp on the anterior side of the cornea, and projecting a slit lamp beam onto the posterior side of the cornea, along with the diffused light from the posterior side of the cornea.

19. A method of examining donor corneas, as recited in claim 13, in which the image received in the viewing means further comprises the creation of a digital image, and transmitting said image to a monitor.
